(19) United States
(12) Patent Application Publication
(10) Pub. No.: US 2004/0063706 A1
(43) Pub. Date: Apr. 1, 2004

(54) Process for the preparation of triazinylaminostilbene-disulphonic acid compounds

(76) Inventors: Georges Metzger, Moernach (FR); Dieter Reinehr, Kandern (DE); Hanspeter Sauter, Schopfheim (DE); Helena Dbaly, Muttenz (CH)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/250,843

(22) PCT Filed: Jan. 7, 2002
(86) PCT No.: PCT/EP02/00070

(30) Foreign Application Priority Data
Jan. 12, 2001 (EP) 01810028.9

(51) Int. Cl. A61K 31/53; C 07D 4 3/02

(52) U.S. Cl. 514/241; 544/193.2

(57) ABSTRACT

A process for the preparation of a 4,4′-bistriazinylamino-stilbene-2,2′-disulphonic acid compound of formula (I) in which, independently, each R₁ represents —NH₂, —NH(C₁-c₁₀alkyl), —N(C₁-C₁₀alkyl)₂, —NH(C₂-C₄hydroxyalkyl), —NH(C₂-C₄hydroxyalkyl)₂, —N(C₁-C₁₀alkyl)(C₂-C₄hydroxyalalkyl), —NH(C₁-C₄alkoxy-C₂-C₄hydroxyalkyl), —N(C₁-C₄alkoxy-C₂-C₆hydroxyalkyl)₂, —N(C₁-C₄alkoxy-C₂-C₄hydroxyalkyl)(C₁-C₁₀alkyl), —N(C₁-C₄alkoxy-C₂C₄hydroxyalkyl)(C₂-C₄hydroxyalkyl), —NH(C₁-C₄alkoxy-C₁-C₄alkoxy-C₁-C₄-alkyl), —N(C₁-C₄alkoxy-C₁-C₄alkoxy-C₁-C₄-alkyl)₂, —N(C₁-C₄alkoxy-C₁-C₄alkoxy-C₁-C₄alkyl)(C₁-C₆alkyl), —N(C₁-C₆alkoxy-C₁-C₄alkoxy-C₂-C₄alkyl)(C₂-C₄hydroxyalkyl)-NH(C₅-C₇cycloalkyl), —N(C₅-C₇cycloalkyl)₂, —NH(C₆-C₁₀aryl), NH(C₇-C₁₃aralkyl) or a morpholino, piperidino or pyrrolidino residue; M represents H, Na, Li, K, Ca, Mg, ammonium, or ammonium that is mono-, di-, tri- or tetrasubstituted by C₁C₄alkyl, C₂-C₄hydroxyalkyl or a mixture thereof, characterized by reacting a compound of the formula (II) in which R₂ represents —C₁-C₁₀alkyl, which is substituted or unsubstituted, and X represents halogen, with at least 4 moles of an amine of formula R₁H(3) or mixtures thereof.

The present invention relates to a process for the preparation of 4,4′-bistriazinylaminostilbene-2,2′-disulphonic acid compounds.

In U.S. Pat. No. 5,939,379 there are described new compounds of the formula

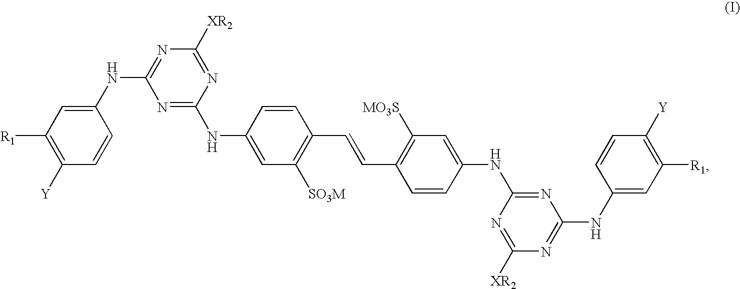

in which Y may be a group of the formula —C(═O)NR₃R₄ in which R₃ and R₄ are hydrogen or C₁-C₄alkyl, X may be NH and R₂ is C₁-C₄alkyl or phenyl, which are useful as ultra-violet absorbing agents (UVA's) and as fluorescent whitening agents (FWA's). The compounds of formula (I) are produced by reacting cyanuric chloride with an aminostilbene-disulphonic acid, an aniline derivative capable of introducing the group

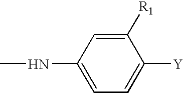

and with a compound capable of introducing the group —XR₂, in any desired sequence. However, this process of preparation suffers from the disadvantage that the above aniline derivatives are not readily available, being both difficult to prepare and also costly.

It has now, surprisingly, been found that compounds of the formula

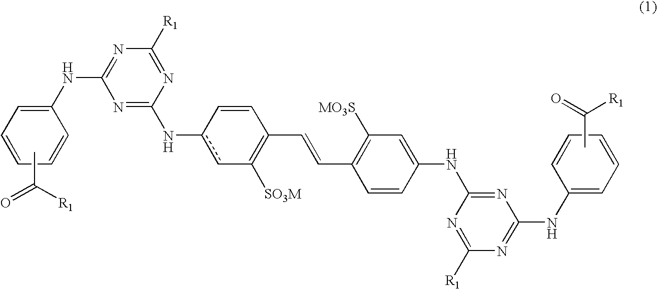

in which, independently, each R₁ represents —NH₂, —NH(C₁-C₁₀alkyl), —N(C₁-C₁₀alkyl)₂, —NH(C₂-C₄hydroxyalkyl), —N(C₂-C₄hydroxyalkyl)₂, —N(C₁-C₁₀alkyl)(C₂-C₄hydroxyalkyl), —NH(C₁-C₄alkoxy-C₂-C₄ hydroxyalkyl), —N(C₁-C₄alkoxy-C₂-C₄ hydroxyalkyl)₂, —N(C₁-C₄alkoxy-C₂-C₄ hydroxyalkyl) (C₁-C₁₀alkyl), —N(C₁-C₄alkoxy-C₂-C₄hydroxyalkyl)(C₂-C₄hydroxyalkyl), —NH(C₁-C₄alkoxy-C₁-C₄alkoxy-C₁-C₄-alkyl), —N(C₁-C₄alkoxy-C₁-C₄alkoxy-C₁-C₄-alkyl)₂, —N(C₁-C₄alkoxy-C₁-C₄alkoxy-C₁-C₄-alkyl)(C₁-C₄alkyl), —N(C₁-C₄alkoxy-C₁-C₄alkoxy-C₂-C₄-alkyl)(C₂-C₄hydroxyalkyl)-NH(C₅-C₇cycloalkyl), —N(C₅-C₇cycloalkyl)₂, —NH(C₆-C₁₀aryl), NH(C₇-C₁₃aralkyl) or a morpholino, piperidino or pyrrolidino residue; M represents H, Na, Li, K, Ca, Mg, ammonium, or ammonium that is mono-, di-, tri- or tetrasubstituted by C₁-C₄alkyl, C₂-C₄hydroxyalkyl or a mixture thereof, may be readily obtained in high purity and in excellent yields by reacting a compound of the formula

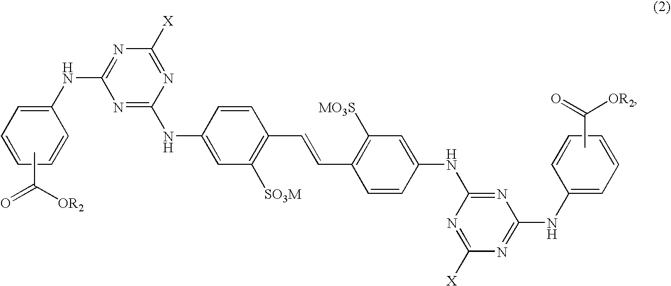

in which R₂ represents —C₁-C₁₀alkyl, which is substituted or unsubstituted, and X represents halogen, with at least 4 moles of an amine of formula R₁H (3) or mixtures thereof.

The process of the invention Is particularly suitable for the preparation of compounds of formula (1) in which each of the R₁ groups are identical and, furthermore is particularly suited to the preparation of a compound of formula

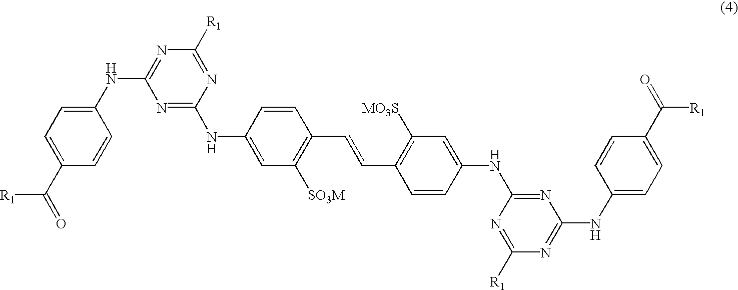

and also for the preparation of a compound of formula

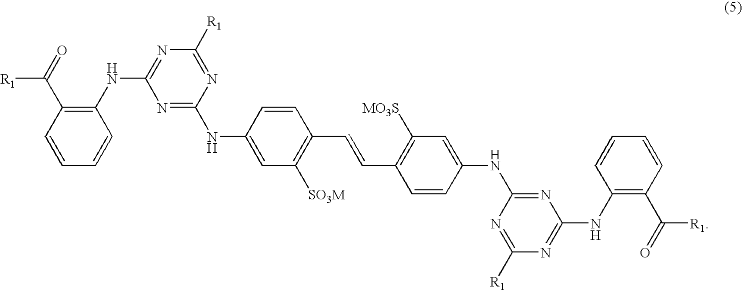

Preferable starting materials of the formula (2) are those in which R₂ is O₁—C₄alkyl, especially methyl or ethyl, and X is fluorine or, especially, chlorine.

The process according to the invention is particularly suitable for the preparation of compounds of formula (1) and, more especially, compounds of the formula (4) and those of the formula (5), in which R₁ is, —NH(C₁-C₄alkyl), —N(C₁-C₄alkyl)₂, —NH(C₂-C₄hydroxyalkyl) or —N(C₂-C₄hydroxyalkyl)₂ and M is hydrogen, K or Na and, most especially, compounds of the formula (4), in which R₁ is mono- or dimethylamino, mono- or diethylamino, or mono- or dihydroxyethylamino.

Within the scope of the compounds of formulae (1) to (5), as C₁-C₁₀alkyl, there are defined methyl, ethyl, n-propyl, isopropyl, n-butyl, sec-butyl or tert-butyl, pentyl, hexyl, heptyl, nonyl or decyl, as C₂-C₄hydroxyalkyl, hydroxyethyl, hydroxypropyl or hydroxybutyl and as C₁-C₄alkoxy, methoxy, ethoxy, n-propoxy, isopropoxy, n-butoxy, sec-butoxy or tert-butoxy. As C₅-C₇cycloalkyl, there are defined cyclopentyl, cyclohexyl or cycloheptyl, whilst C₆-C₁₀aryl may be phenyl, which is unsubstituted or substituted by C₁-C₄alkyl or C₁-C₄alkoxy or, alternatively, naphthalene and C₇-C₁₃aralkyl is, for example, substituted or unsubstituted benzyl, phenethyl, naphthylmethyl or naphthylethyl.

Halogen, in compound of formula (2), is bromine, fluorine or, especially, chlorine.

The amount of the amine of formula (3) to be reacted with the compound of formula (2) should correspond to at least stoichiometric quantities, i.e. 4 moles per mole, based on the molar amount of the compound of formula (2). However, since the amine may also function as an acid acceptor, it is preferably used in excess, whereby it may further function as solvent for the reaction. Alternatively, a solvent may also be advantageously employed for carrying out reaction, as may also a secondary base, different to that of the amine of formula (3), as acid acceptor during reaction. Clearly, where mixtures of amines of formula (3) are employed, the resulting reaction product will consist of corresponding mixtures of compounds of formula (1).

Suitable solvents which may be employed for reaction of the compound of formula (2) with the amine of formula (3) are, for example, dipolar aprotic solvents such as dimethylformamide, dimethylsulphoxide, N-methylpyrrolidone or tetramethyl urea, alcohols such as methyl alcohol, ethyl alcohol, n- or isopropanol or a butyl alcohol, ketones such as acetone or methyl ethyl ketone or, especially, water, or mixtures thereof.

Suitable secondary bases which may be employed as acid acceptors for the reaction are, for example, tertiary organic amines such as triethylamine, tripropylamine or tributylamine, or inorganic bases such as potassium or sodium carbonates, bicarbonates or hydroxides and also other inorganic acid acceptors such as calcium or magnesium oxides.

The temperature at which reaction of the compound of formula (2) with the amine of formula (3) is carried out may vary over a large range, for example between approximately 20° C. and approximately 200° C., depending upon the amine component to be employed. However, temperatures within the range of from about 50° C. to about 150° C. and, in particular, within the range of from about 80° C. to about 130° C.

Depending upon the reaction temperature and the amine compound of formula (3) employed, it may be advantageous to carry out reaction at atmospheric pressure, under reduced pressure or under higher pressure, in which case a suitable pressure vessel such as an autoclave should be employed.

In a further aspect of the invention, by the addition of a suitable formulation agent to the reaction mixture, which may, furthermore, serve as solvent for the reaction, it may be possible to obtain stable liquid formulations directly from the reaction mixture. The desired concentration of the formulation may then be simply obtained, either by concentration of the reaction mixture or by dilution, for example, with water.

Suitable formulation agents which may be used for this aspect of the invention are, for example, polyhydroxy compounds such as glycerin or ethylene or propylene glycol or, in particular, polyethylene glycols such as polyethylene glycol 150, 200, 600, 1500 and, especially, polyethylene glycol 300.

The starting materials of formulae (2) and (3) are known compounds which are readily available or may be obtained by known methods.

The following Examples further illustrate the present invention without intending to be restrictive in nature; parts and percentages are by weight, unless otherwise stated.

### EXAMPLE 1

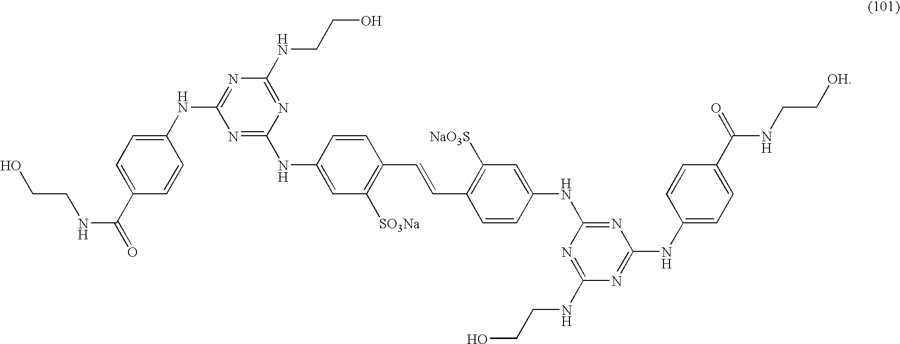

2.0 g of the compound of formula

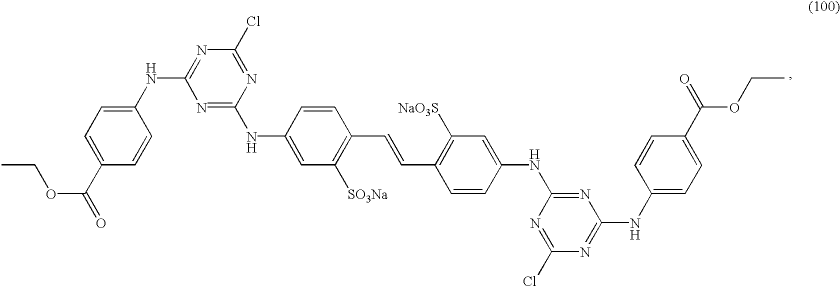

prepared according to U.S. Pat. No. 5,744,599, with an active content of 85%, are added to a mixture of 1.5 g of ethanolamine and 1.0 g of polyethylene glycol 300. The mixture is heated to a temperature of 120° C., whereby at 100° C. a clear solution results. After stirring for 4 hours at 120° C., the mixture is cooled to 80° C. and 3.7 g of water are added. There results a stable formulation containing

22% of the compound of formula (101),

14% ethanolamine,

12.5% polyethylene glycol 300,

2.5% sodium chloride and

49% water.

### EXAMPLE 2

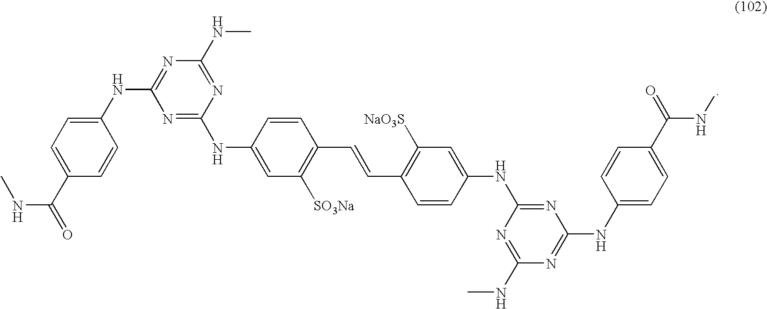

2.5 g of the compound of formula (100) with an active content of 85% are added to 16.0 g of 40% aqueous methylamine solution and placed in a glass autoclave. After closure of the autoclave, the reaction mixture is stirred for 3 hours at 90° C., cooled and treated with 2.2 ml of 2N sodium hydroxide solution. The precipitated solids are filtered with suction and dried to yield 80% of the compound of formula (102), calculated on the amount of compound (100) employed, which, according to HPLC and UV spectrum, is identical with an authentic sample, prepared as described in U.S. Pat. No. 5,939,379.

1. A process for the preparation of a 4,4′-bistriazinylamino-stilbene-2,2′-disulphonic acid compound of the formula 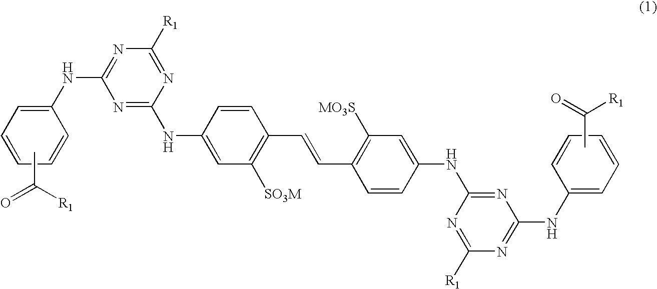
in which, independently, each
R₁ represents —NH₂, —NH(C₁-C₁₀alkyl), —N(C₁-C₁₀alkyl)₂, —NH(C₂-C₄hydroxyalkyl), —N(C₂-C₄hydroxyalkyl)₂, —N(C₁-C₁₀alkyl)(C₂-C₄hydroxyalkyl), —NH(C₁-C₄alkoxy-C₂-C₄ hydroxyalkyl), —N(C₁-C₄alkoxy-C₂-C₄ hydroxyalkyl)₂, —N(C₁-C₄alkoxy-C₂-C₄ hydroxyalkyl) (C₁-C₁₀alkyl), —N(C₁-C₄alkoxy-C₂-C₄ hydroxyalkyl)(C₂-C₄hydroxyalkyl), —NH(C₁-C₄alkoxy-C₁-C₄alkoxy-C₁-C₄-alkyl), —N(C₁-C₄alkoxy-C₁-C₄alkoxy-C₁-C₄-alkyl)₂, —N(C₁-C₄alkoxy-C₁-C₄alkoxy-C₁-C₄-alkyl)(C₁-C₄alkyl), —N(C₁-C₄alkoxy-C₁-C₄alkoxy-C₂-C₄-alkyl)(C₂-C₄ hydroxyalkyl)-NH(Cs-C₇cycloalkyl), —N(C₅-C₇cycloalkyl)₂, —NH(C₆-C₁₀aryl), NH(C₇-C₁₃aralkyl) or a morpholino, piperidino or pyrrolidino residue;
M represents H, Na, Li, K, Ca, Mg, ammonium, or ammonium that is mono-, di-, tri- or tetrasubstituted by C₁-C₄alkyl, C₂-C₄hydroxyalkyl or a mixture thereof,
characterized by reacting a compound of the formula 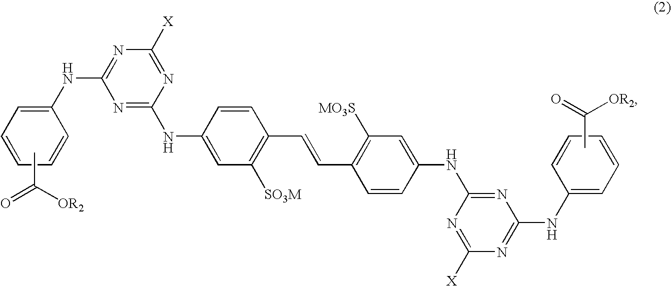
in which
R₂ represents —C₁-C₁₀alkyl, which is substituted or unsubstituted, and
X represents halogen, with at least 4 moles of an amine of formula
R₁H (3) or mixtures thereof.

2. A process according to claim 1 in which each of the R₁ groups are identical.

3. A process according to claims 1 or 2 for the preparation of a compound of formula

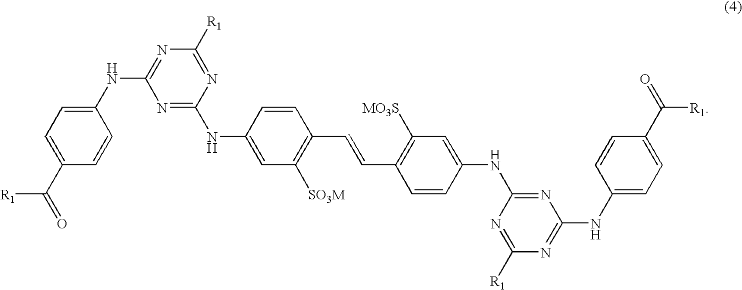

4. A process according to claims 1 or 2 for the preparation of a compound of formula

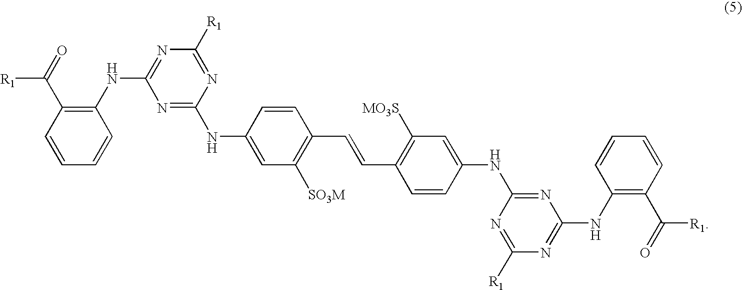

5. A process according to any one of the preceding claims in which
R₂ is C₁-C₄alkyl and
X is chlorine.

6. A process according to any one of the preceding claims in which
R₁ is, —NH(C₁-C₄alkyl), —N(C₁-C₄alkyl)₂, —NH(C₂-C₄hydroxyalkyl) or —N(C₂-C₄hydroxyalkyl)₂ and
M is hydrogen, K or Na.

7. A process according to claim 6 for the preparation of the compound of formula (4).

8. A process according to claim 6 for the preparation of the compound of formula (5).

9. A process according to any one of the preceding claims in which the reaction is carried out in the presence of a basic compound other than the amine R₁H.

10. A process according to any one of the preceding claims in which the reaction is carried out in the presence of a formulating agent, especially a polyethylene glycol.
